(19) United States
(12) Patent Application Publication
(10) Pub. No.: US 2016/0254098 A1
(43) Pub. Date: Sep. 1, 2016

(54) METHOD FOR PREPARING IONIC LIQUID HAVING CARBOXYLIC ACID ANION USING MICROREACTOR

(71) Applicant: CHEMTECH RESEARCH INCORPORATION, Gyeonggi-do (KR)

(72) Inventors: Jung Bok RYU, Chuncheon-si (KR); Duck Soo YUK, Seoul (KR); Kuk Jin JANG, Gwangyang-si (KR); So Kyoung JOO, Seongnam-si (KR); Wan Joo KIM, Seoul (KR); Jin Young JANG, Namyangju-si (KR); Tae Seop HWANG, Cheonan-si (KR)

(21) Appl. No.: 14/904,427

(22) PCT Filed: Jul. 11, 2013
(86) PCT No.: PCT/KR2013/006213

§ 371 Date: May 2, 2016

(51) Int. Cl. H01G 9/035; C07C 209/68; C07D 207/06; C07D 213/20; C07D 233/58; C07C 51/41

(52) U.S. Cl. CPC H01G 9/035; C07D 233/58; C07C 51/412; C07D 207/06; C07D 213/20; C07C 209/68

(57) ABSTRACT

The present invention relates to a method for preparing an ionic liquid having a carboxylic acid anion using a microreactor. More specifically, the present invention relates to a method for preparing, with high efficiency, an ionic liquid having a carboxylic acid anion as shown in FIG. 1, by having sodium butanoate, sodium 2-ethylhexanoate, or sodium octanoate undergo a substitution reaction with 1-alkyl-3-methylimidazolium, 1,1-alkylmethylpyrrolidinium, 1,2-dimethyl-3-alkylimidazolium, 1-alkyl-3-methylpyridinium, or tetramethylammonium, each of which being a cation. The ionic liquid prepared according to the present invention has high purity, containing residual halide at less than 10 ppm, and has high electrical conductivity, and therefore is capable of being used as an electrolyte or for a condenser.

### FIELD OF THE INVENTION

The present invention relates to a method for preparing an ionic liquid having a carboxylic acid anion using a microreactor. More specifically, the present invention relates to a method for preparing, with high efficiency, an ionic liquid having a carboxylic acid anion as shown in FIG. 1, by having sodium butanoate, sodium 2-ethylhexanoate, or sodium octanoate undergo a substitution reaction with 1-alkyl-3-methylimidazolium, 1,1-alkylmethylpyrrolidinium, 1,2-dimethyl-3-alkylimidazolium, 1-alkyl-3-methylpyridinium, or tetramethylammonium, each of which being a cation.

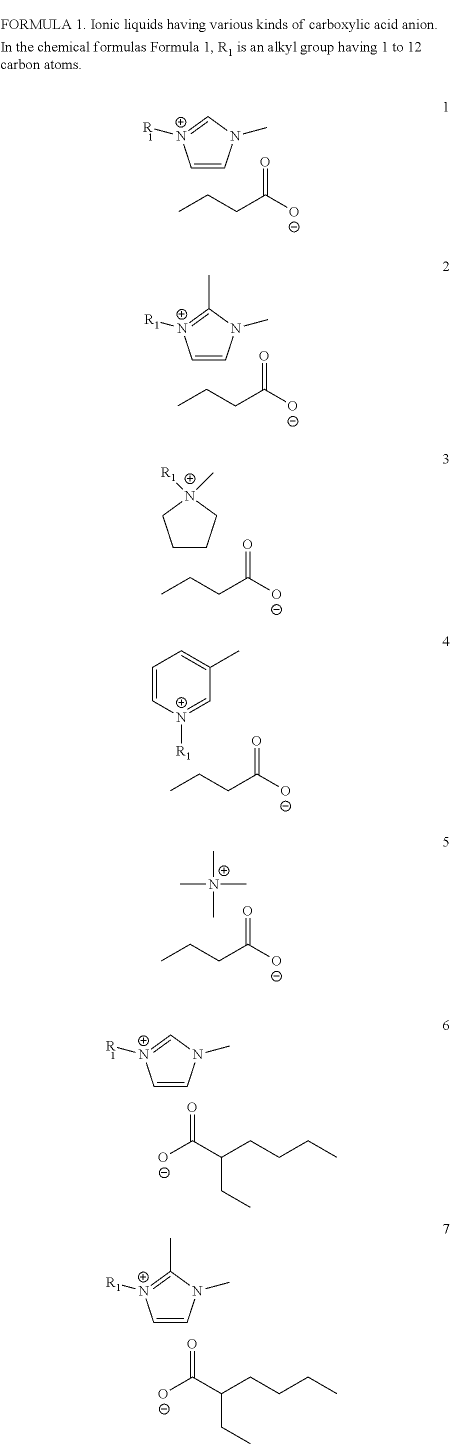

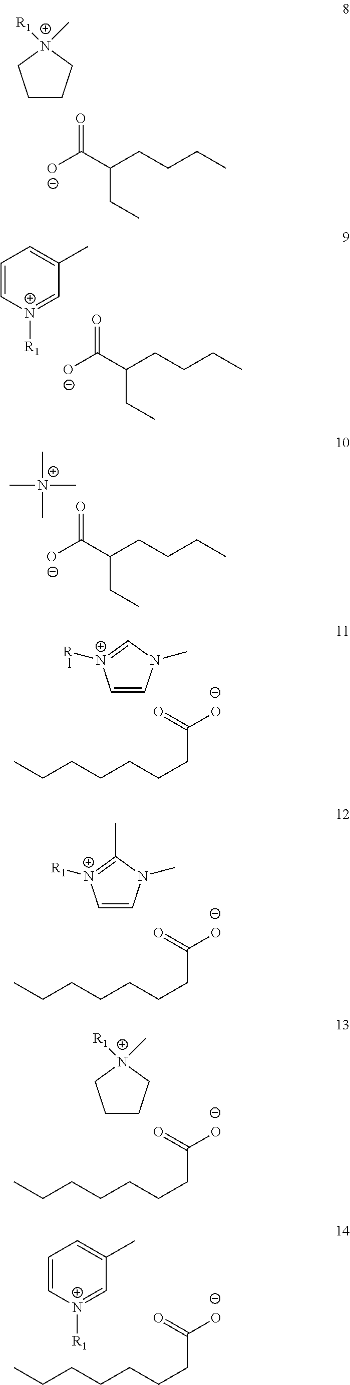

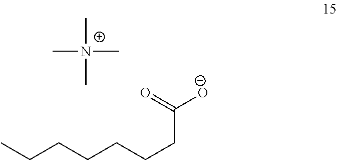

In the chemical formulas of Formula 1, R₁ is an alkyl group having 1 to 12 carbon atoms.

### BACKGROUND OF THE INVENTION

Ionic liquids have a high heat-resistant temperature, are non-flammable and tend to have a low water solubility unlike other materials having conventional ions and also show a good solubility in organic solvents. In addition, ionic liquids have an excellent conductivity because the electrons vigorously move.

Due to these properties, ionic liquids are extensively used as green solvents, lithium secondary battery, organic solar cell and electrolytes for a capacitor in various fields including organic synthesis, electrochemistry, biotechnology, chemical engineering and separation process. In these cases, contaminants and purity are critical factors. Contaminants, such as residual halide, starting materials remaining after reactions, degradation products or water generally increase a resistance during the process of an electrochemical reaction. As reported in Electrochemical Society Proceeding, Volumes, 99-41, when the residual halide reacted with liquid hydrogen is used or Suzuki reaction occurs, the reactions are interrupted. Thus, an ionic liquid having high purity is recognized to be important.

A conventional method for preparing an ionic liquid by using alkyl halide is cost consuming and difficult to mass produce ionic liquids. Therefore, such method is not acknowledged to be effective or economical. In addition, there is a method of using an acid (HA) to volatilize into hydrogen, but this method causes a corrosion and also emits harmful gases and thus is hard to be used. Besides, methods for removing halides by using silver nitrate to remove halogen ions as insoluble silver halides, or by using lead salts to remove halogen ions as insoluble lead halides have been reported, but metal salts are expensive and emit wastes containing harmful metals. Therefore, these methods are not recognized to be effective.

In particular, bromoethane, chlorobutane and the like have been conventionally used in the mass production of ionic liquids. However, intermediates including halogen compounds generate heat and pressure; are very unstable because the compounds absorb moisture in the air and decompose; require a long reaction time more than 24 hours; and produce an anion of ionic liquids containing halogen and thus it is hard to lower the level of residual halide below 500 ppm. Further, when an anion of ionic liquids is methyl sulfate or ethyl sulate, it is hard to neutralize pH of the ionic liquids and also the ionic liquids are corrosive. Therefore, the present method for preparing ionic liquids having carboxylic acid anion has been invented.

### DETAILED DESCRIPTION OF THE INVENTION

### Technical Objects

The object of the present invention is to provide a method for preparing, with high efficiency, an ionic liquid having a carboxylic acid anion, by having sodium butanoate, sodium 2-ethylhexanoate, or sodium octanoate undergo a substitution reaction with 1-alkyl-3-methylimidazolium, 1,1-alkylmethylpyrrolidinium, 1,2-dimethyl-3-alkylimidazolium, 1-alkyl-3-methylpyridinium, or tetramethylammonium, each of which being a cation.

### SUMMARY OF THE INVENTION

The ionic liquids having carboxylic acid anion was synthesized by having sodium butanoate, sodium 2-ethylhexanoate or sodium octanoate undergo a reaction with a halogen salt of 1-alkyl-3-methylimidazolium, 1,1-alkylmethylpyrrolidinium, 1,2-dimethyl-3-alkylimidazolium, 1-alkyl-3-methylpyridinium, or tetramethylammonium as starting materials to substitute the anion of the starting materials with butanoate, hexanoate or octanoate. The alkyl group of 1-alkyl-3-methylimidazolium, 1,1-alkylmethylpyrrolidinium, 1,2-dimethyl-3-alkylimidazolium, 1-alkyl-3-methylpyridinium has 1 to 12 carbon atoms and the halogen is fluorine, chlorine, bromine or iodine. The substitution reaction is carried out by using a bench reaction or microreactor.

By using the novel method for preparing ionic liquid having carboxylic acid anion, a synthesis yield and a chemical purity can be improved; the unit cost of production can be reduced; and an ionic liquid having high purity which is applicable as an electrolyte can be provided by selecting the ionic liquid having a high thermal stability and an electrical conductivity.

Synthesis yield, content of halide, electrical conductivity and thermal stability (TGA) are evaluated for the compounds produced according to the method of the present invention.

### Technical Effects

The method for preparing an ionic liquid according to the present invention uses a carboxylic acid anion, such as sodium butanoate, sodium 2-ethylhexanoate, or sodium octanoate. Therefore, the method is environmentally friendly because water can be used as a solvent for the anion substitution reaction and also the time for the substitution reaction can be shortened less than one hour.

In addition, by using the method according to the present invention, a high-purity ionic liquid having residual halide less than 20 ppm can be produced. Therefore, the method for preparing an ionic liquid according to the present invention has advantages of maximizing an efficiency and an economical efficiency since there are only few residual halide to be removed.

Further, the compounds including butanoate, hexanoate and octanoate according to the present invention are stable and also the time for the anion substitution reaction is shorter (less than one hour) than that of the conventional method for preparing the intermediates. Moreover, the ionic liquids prepared by the method according to the present invention are pure and contain residual halides less than 10 ppm, and thus can be used as electrolytes and capacitors due to their good electrical conductivity.

### BRIEF DESCRIPTION OF THE DRAWINGS

FIG. 1 shows the whole structure of the microreactor synthesizer.

FIG. 2 illustrates a schematic diagram of the synthesis which shows the process that reagents A and B are allowed to flow to microreactor through a cylinder pump and pass a micromixer, and then the target compounds are synthesized.

FIG. 3 represents Y-type, Helix-type and Static-type micromixers.

FIG. 4 shows a halide measuring equipment, 716 DMS Titrino ion analyzer.

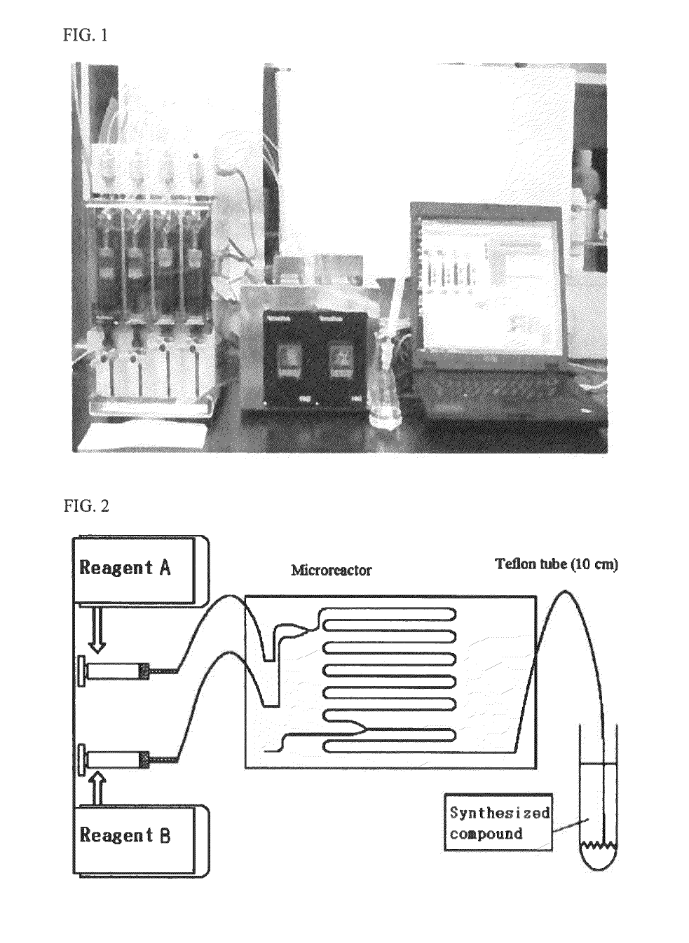

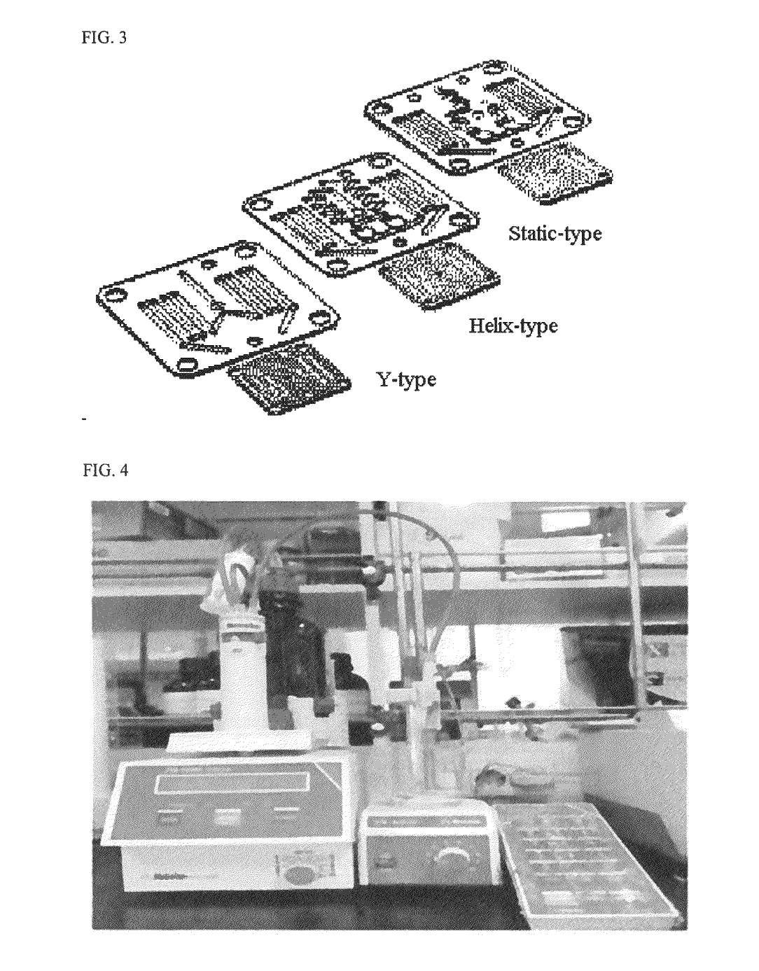

### BEST MODE OF THE INVENTION

In the present invention, a high-purity ionic liquid having a carboxylic acid anion is provided. The method according to the present invention comprises a step of having sodium butanoate, sodium 2-ethylhexanoate or sodium octanoate undergo a reaction with a halogen salt of 1-alkyl-3-methylimidazolium, 1,1-alkylmethylpyrrolidinium, 1,2-dimethyl-3-alkylimidazolium, 1-alkyl-3-methylpyridinium, or tetramethylammonium as starting materials to substitute the anion of the starting materials with butanoate, hexanoate or octanoate. The alkyl group of 1-alkyl-3-methylimidazolium, 1,1-alkylmethylpyrrolidinium, 1,2-dimethyl-3-alkylimidazolium, 1-alkyl-3-methylpyridinium has 1 to 12 carbon atoms and the halogen is fluorine, chlorine, bromine or iodine. In addition, the substitution reaction is carried out by using a bench reaction or microreactor.

### MODE OF THE INVENTION

The following examples is to more specifically explain the present invention, but the present invention does not limited thereto.

### <Used Apparatus>

KeyChem-L (YMC, Japan) was used as a microreactor and MRSY04-40 was used as a cylinder pump. A Helix-type micromixer was used, which has a good efficiency for heat exchange and is suitable for organic synthesis.

### <Measuring Residual Halides>

Residual halides were measured using 716 DMS Titrino ion analyzer (Metrohm) according to an analytical method based on the standard measurement method.

### <Measuring Electrical Conductivity>

856 Conductivity Module (Metrohm) was used, and residual halides were measured according to an analytical method based on the standard measurement method.

### <TGA Analysis>

PERKIN ELMER TGA7 model was used. Korean Institute of Industrial Technology was requested to measure with the analytical condition of 30° C., (5 min)→10° C./min→800° C.

### EXAMPLE

### Example 1

### Synthesis of 1-butyl-3-methylimidazolium butanoate

5.0 g of 1-butyl-3-methylimidazolium chloride (0.028 mol) was dissolved in 10 g water and then allowed to flow to a microreactor adjusted at 40° C. through a cylinder pump at a flow rate of 300 μl/min. 3.78 g of sodium butanoate (0.034 mol) was dissolved in 10 g of water and then allowed to flow to a microreactor adjusted at 40° C. through a cylinder pump at a flow rate of 281 μl/min. The solutions that passed through the microreactor were collected and then concentrated under reduced pressure to give 6.15 g 1-butyl-3-methylimidazolium butanoate as a pale white solid (97%). The analysis result of the obtained ionic liquid was as follows:

¹H-NMR (DMSO, 400 MHz) δ: 7.32 (d, 1H), 7.28 (d, 1H), 4.03 (t, 2H), 3.74 (s, 3H), 1.99 (t, 2H), 1.68 (q, 2H), 1.14 (t, 3H), 0.73 (t, 3H), residual halide: 5 ppm, electrical conductivity (2.5° C.): 1.187 mS/cm, thermal stability (TGA): 247° C.

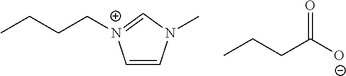

### Example 2

### Synthesis of 1,2-dimethyl-3-butylimidazolium butanoate

5.0 g of 1,2-dimethyl-3-butylimidazolium iodide (0.018 mol) was dissolved in 20 g of water and then allowed to flow to a microreactor adjusted at 40° C. through a cylinder pump at a flow rate of 300 μl/min. 2.13 g sodium butanoate (0.021 mol) was dissolved in 20 g of water and then allowed to flow to a microreactor adjusted at 40° C. through a cylinder pump at a flow rate of 288 μl/min. The solutions that passed though the microreactor were collected and then concentrated under reduced pressure to give 4.09 g of 1,2-dimethyl-3-butylimidazolium butanoate as a while solid (94%). The analysis result of the obtained ionic liquid was as follows:

¹H-NMR (DMSO, 400MHz) δ: 7.61 (d, 1H), 7.59 (d, 1H), 4.07 (m, 2H), 3.71 (s, 3H), 2.60 (s, 3H), 2.35 (s, 2H), 1.66 (t, 2H), 1.64 (t, 2H), 1.23 (q, 2H), 1.21 (q, 2H), 1.21, 0.84 (t, 32H), 0.77 (t, 3H), residual halide: 3 ppm, electrical conductivity (25° C.): 2.032 mS/cm, thermal stability (TGA): 287° C.

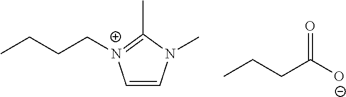

### Example 3

### Synthesis of 1,1-butylmethylpyrrolidinium butanoate

2.0 g of 1,1-butylmethylpyrrolidinium bromide (0.009 mol) was dissolved in 20 g of water and then allowed to flow to a microreactor adjusted at 40° C. through a cylinder pump at a flow rate of 300 μl/min. 1.14 g of sodium butanoate (0.010 mol) was dissolved in 20 g of water and then allowed to flow to a microreactor adjusted at 40° C. through a cylinder pump at a flow rate of 297 μl/min. The solutions that passed through the microreactor were collected and then concentrated under reduced pressure to give 1.78 g of 1,1-butylmethylpyrrolidinium butanoate as a white solid (97%). The analysis result of the obtained ionic liquid was as follows:

¹H-NMR (DMSO, 400 MHz) δ: 4.63 (s, 2H), 3.37 (m, 2H), 3.18 (m, 2H), 2.91 (s, 2H), 2.08 (t, 2H), 2.00 (t, 2H), 1.42 (m, 3H), 1.30 (s, 2H), 1.24 (s, 2H), 0.81 (t, 3H), 0.75 (t, 3H), residual halide: 1 ppm, electrical conductivity (250° C.): 1.156 mS/cm, thermal stability (TGA): 243° C.

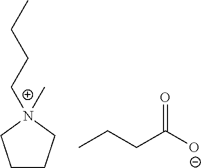

### Example 4

### Synthesis of 1-butyl-3-methylpyridinium butanoate

5.0 g of 1-butyl-3-methylpyridinium chloride (0.027 mol) was dissolved in 20 g of water and then allowed to flow to a microreactor adjusted at 40° C. through a cylinder pump at a flow rate of 300 μl/min. 3.10 g of sodium butanoate (0.028 mol) was dissolved in 20 g of water and then allowed to flow to a microreactor adjusted at 40° C. through a cylinder pump at a flow rate of 293 μl/min. The solutions that passed through the microreactor were collected and then concentrated under reduced pressure to give 5.84 g of 1-butyl-3-methylpyridinium butanoate as a pale white solid (91%). The analysis result of the obtained ionic liquid was as follows:

¹H-NMR (DMSO, 400 MHz) δ: 8.51 (s, 1H), 8.50 (d, 1H), 8.21 (d, 1H), 7.74 (m, 1H), 4.60 (t, 2H), 4.40 (s, 3H), 1.92 (t, 2H), 1.80 (q, 2H), 1.38 (q, 2H), 1.16 (m, 2H), 0.78 (t, 3H), 0.73 (t, 3H), residual halide: 7 ppm, electrical conductivity (25° C.): 1.159 mS/cm, thermal stability (TGA): 257° C.

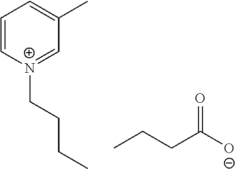

### Example 5

### Synthesis of tetramethylammonium butanoate

5.0 g of tetramethylammonium chloride (0.046 mol) was dissolved in 20 g of water and then allowed to flow to a microreactor adjusted at 40° C. through a cylinder pump at a flow rate of 300 μl/min. 6.03 g of sodium butanoate (0.055 mol) was dissolved in 20 g of water and then allowed to flow to a microreactor adjusted at 40° C. through a cylinder pump at a flow rate of 296 μl/min. The solutions that passed through the microreactor were collected and then concentrated under reduced pressure to give 8.52 g of tetramethylammonium butanoate as a pale white solid (96%). The analysis result of the obtained ionic liquid was as follows:

¹H-NMR (DMSO, 400 MHz) δ: 3.08 (s, 12H), 1.78 (q, 2H), 1.64 (t, 2H), 1.37 (q, 2H), 0.75 (t, 3H), residual halide: 8 ppm, electrical conductivity (25° C.): 0.829 mS/cm, thermal stability (TGA): 262° C.

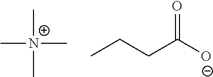

### Example 6

### Synthesis of 1,1-butylmethylpyrrolidinium 2-ethylhexanoate

0.2 g of 1,1-butylmethylpyrrolidinium bromide (0.0009 mol) was dissolved in 15 g of water and then allowed to flow to a microreactor adjusted at 70° C. through a cylinder pump at a flow rate of 150 μl/min. 0.15 g of sodium 2-ethylhexanoate (0.0011 mol) was dissolved in 15 g of water and then allowed to flow to a microreactor adjusted at 70° C. through a cylinder pump at a flow rate of 150 μl/min. The solutions that passed through the microreactor were collected and then concentrated under reduced pressure to give 0.215 g 1,1-butylmethylpyrrolidinium 2-ethylhexanoate as a pale yellow solid (93%). The analysis result of the obtained ionic liquid was as follows:

¹H-NMR (acetone-d₆, 400 MHz) δ: 3.81 (m, 4H), 3.68 (m, 2H), 3.28 (s, 3H), 2.26 (m, 4H), 1.99 (m, 1H), 1.85 (m, 2H), 1.54 (m, 2H), 1.42 (m, 2H), 1.26 (m, 6H), 0.98 (t, 3H), 0.84 (t, 6H), residual halide: 8 ppm, electrical conductivity (25° C.): 4.561 mS/cm, thermal stability (TGA): 246° C.

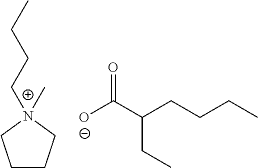

### Example 7

### Synthesis of tetramethylammonium 2-ethylhexanoate

0.2 g of tetramethylammonium chloride (0.002 mol) was dissolved in 10 g of water and then allowed to flow to a microreactor adjusted at 70° C. through a cylinder pump at a flow rate of 150 μl/min. 0.30 g of sodium 2-ethylhexanoate (0.002 mol) was dissolved in 10 g of water and then allowed to flow to a microreactor adjusted at 70° C. through a cylinder pump at a flow rate of 150 μl/min. The solutions that passed through the microreactor were collected and then concentrated under reduced pressure to give 0.335 g of tetramethylammonium 2-ethylhexanoate as a white solid (98%). The analysis result of the obtained ionic liquid was as follows:

¹H-NMR (acetone-d₆, 400 MHz) δ: 3.28 (s, 12H), 2.01 (m, 1H), 1.56 (m, 2H), 1.29 (m, 6H), 0.85 (m, 6H), residual halide: 6 ppm, electrical conductivity (25° C.): 0.388 mS/cm, thermal stability (TGA): 460° C.

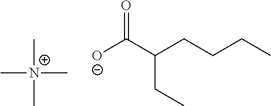

### Example 8

### Synthesis of 1-butyl-3-methylimidazolium octanoate

0.2 g of 1-butyl-3-methylimidazolium chloride (0.001 mol) was dissolved in 10 g of water and then allowed to flow to a microreactor adjusted at 70° C. through a cylinder pump at a flow rate of 150 μl/min. 0.17 g of sodium octanoate (0.001 mol) was dissolved in 10 g of water and then allowed to flow to a microreactor adjusted at 70° C. through a cylinder pump at a flow rate of 150 μl/min. The solutions that passed through the microreactor were collected and then concentrated under reduced pressure to give 0.31 g of 1-butyl-3-methylimidazolium octanoate as a pale white solid (91%). The analysis result of the obtained ionic liquid was as follows:

¹H-NMR (methanol, d₄, 400 MHz) δ: 7.54 (d, 1H), 7.47 (d, 1H), 4.11 (t, 2H), 3.89 (s, 3H), 2.05 (t, 2H), 1.77 (m, 2H), 1.49 (m, 214), 1.31 (m, 2H), 1.21 (m, 8H), 0.89 (t, 3H), 0.79 (t, 3H), residual halide: 4 ppm, electrical conductivity (25° C.) 4.080 mS/cm, thermal stability (TGA): 236° C.

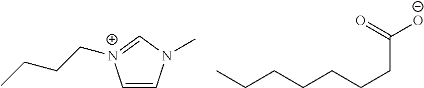

### Example 9

### Synthesis of 1,2-dimethyl-3-ethylimidazolium octanoate

0.2 g of 1,2-dimethyl-3-ethylimidazolium bromide (0.001 mol) was dissolved in 12 g of water and then allowed to flow to a microreactor adjusted at 80° C. through a cylinder pump at a flow rate of 150 μl/min. 0.17 g of sodium octanoate (0.001 mol) was dissolved in 12 g of water and then allowed to flow to a microreactor adjusted at 80° C. through a cylinder pump at a flow rate of 150 μl/min. The solutions that passed through the microreactor were collected and then concentrated under reduced pressure to give 0.25 g of 2-dimethyl-3-ethylimidazolium octanoate as a white solid (93%). The analysis result of the obtained ionic liquid was as follows:

¹H-NMR (methanol-d₄, 400 MHz) δ: 7.51 (d, 1H), 7.45 (d, 1H), 4.18 (m, 2H), 3.79 (s, 3H), 2.60 (s, 3H), 2.12 (t, 2H), 1.57 (m, 2H), 1.43 (t, 3H), 1.29 (m, 8H), 0.87 (t, 3H), residual halide: 2 ppm, electrical conductivity (25° C.) 4.422 mS/cm, thermal stability (TGA): 269° C.

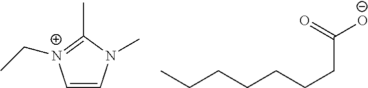

### Example 10

### Synthesis of 1,1-butylmethylpyrrolidinium octanoate

0.2 g of 1,1-butylmethylpyrrolidinium chloride (0.001 mol) was dissolved in 10 g of water and then allowed to flow to a microreactor adjusted at 70° C. through a cylinder pump at a flow rate of 150 μl/min. 0.17 g of sodium 2-octanoate (0.001 mol) was dissolved in 10 g of water and then allowed to flow to a microreactor adjusted at 70° C. through a cylinder pump at a flow rate of 150 μl/min. The solutions that passed through the microreactor were collected and then concentrated under reduced pressure to give 0.265 g of 1,1-butylmethylpyrrolidinium octanoate as a white solid (94%). The analysis result of the obtained ionic liquid was as follows:

¹H-NMR (methanol-d₄, 400 MHz) δ: 152 (m, 4H), 136 (m, 2H), 3.05 (s, 3H), 2.24 (m, 4H), 2.14 (t, 2H), 1.78 (m, 2H), 1.58 (m, 2H), 1.43 (m, 2H), 1.31 (m, 8H), 1.02 (t, 3H), 0.89 (t, 3H), residual halide: 8 ppm, electrical conductivity (25° C.): 2.496 mS/cm, thermal stability (TGA): 208° C.

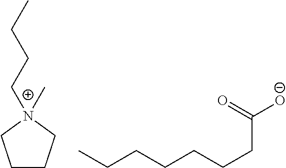

### Example 11

### Synthesis of 1-butyl-3-methylpyridinium octanoate

0.2 g of 1-butyl-3-methylpyridinium chloride (0.001 mol) was dissolved in 10 g of water and then allowed to flow to a microreactor adjusted at 80° C. through a cylinder pump at a flow rate of 150 μl/min. 0.17 g of sodium octanoate (0.001 mol) was dissolved in 10 g of water and then allowed to flow to a microreactor adjusted at 80° C. through a cylinder pump at a flow rate of 150 μl/min. The solutions that passed through the microreactor were collected and then concentrated under reduced pressure to give 0.279 g of 1-butyl-3-methylpyridinium octanoate as a pale brown solid (96%). The analysis result of the obtained ionic liquid was as follows:

¹H-NMR (methanol-d₄, 400 MHz) δ: 8.89 (s, 1H), 8.82 (d, 1H), 8.43 (d, 1H), 8.00 (m, H), 4.60 (t, 2H), 2.58 (s, 3H), 2.16 (t, 2H), 2.01 (m, 2H), 1.62 (m, 2H), 1.57 (m, 2H), 1.44 (m, 8H), 1.02 (t, 3H), 0.91 (t, 3H), residual halide: 9 ppm, electrical conductivity (25° C.): 2.762 mS/cm, thermal stability (TGA): 209° C.

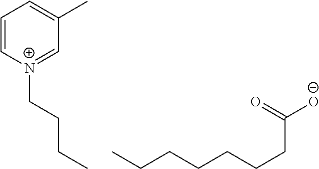

### Example 12

### Synthesis of tetramethylammonium octanoate

0.2 g of tetramethylammonium chloride (0.001 mol) was dissolved in 15 g of water and then allowed to flow to a microreactor adjusted at 70° C. through a cylinder pump at a flow rate of 150 μl/min. 0.17 g of sodium octanoate (0.001 mol) was dissolved in 15 g of water and then allowed to flow to a microreactor adjusted at 70° C. through a cylinder pump at a flow rate of 150 μl/min. The solutions that passed through the microreactor were collected and then concentrated under reduced pressure to give 0.214 g of tetramethylammonium octanoate as a white solid (96%). The analysis result of the obtained ionic liquid was as follows:

¹H-NMR (methanol-d₄; 400 MHz) δ: 3.19 (s, 12H), 2.16 (t, 2H), 1.62 (m, 2H), 1.31 (m, 8H), 0.91 (t, 3H), residual halide: 8 ppm, electrical conductivity (25° C.): 0.794 mS/cm, thermal stability (TGA): 233° C.

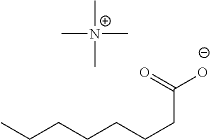

1: A method of preparing a high-purity ionic liquid having carboxylic add anion, comprising
having sodium butanoate, sodium 2-ethylhexanoate or sodium octanoate undergo a reaction with a halogen salt of 1-alkyl-3-methylimidazolium, 1,1-alkylmethylpyrrolidinium, 1,2-dimethyl-3-alkylimidazolium, 1-alkyl-3-methylpyridinium or tetramethylammonium as starting materials to substitute the anion of the starting materials with butanoate, hexanoate or octanoate, wherein the alkyl group of 1-alkyl-3-methylimidazolium, 1,1-alkylmethylpyrrolidinium, 1,2-dimethyl-3-alkylimidazolium, 1-alkyl-3-methylpyridinium has 1 to 12 carbon atoms and the halogen is fluorine, chlorine, bromine or iodine.

2: The method of claim 1, wherein the solvent for the substitution reaction is water.

3: The method of claim 1, wherein the substitution reaction is carried out within one hour.

4: The method of claim 1, wherein the residual halide of the prepared ionic liquid is less than 20 ppm.

5: The method of claim 1, wherein the substitution reaction is carried out by using a bench reaction or a microreactor.
